(19) United States
(12) Patent Application Publication
(10) Pub. No.: US 2024/0257924 A1
(43) Pub. Date: Aug. 1, 2024

(54) CLINICAL TRIAL MATCHING PLATFORM

(71) Applicant: Trial Library, Inc., San Francisco, CA (US)

(72) Inventors: Hala Borno, San Francisco, CA (US); Stephen Buck, San Diego, CA (US); Krunal Patel, Fremont, CA (US); Omar Mehmood, Las Vegas, NV (US); Sylvia Tina Zhang, San Francisco, CA (US); Matthew Adam Oberman, San Francisco, CA (US)

(21) Appl. No.: 18/427,624

(22) Filed: Jan. 30, 2024

Related U.S. Application Data
(60) Provisional application No. 63/442,049, filed on Jan. 30, 2023.

(51) Int. Cl. G16H 10/20

(52) U.S. Cl. CPC G16H 10/20

(57) ABSTRACT

A clinical trial matching platform can have an inventory of clinical trials that are recruiting participants. The clinical trial matching platform can present lists of such clinical trials to medical providers, who can agree to perform pre-screening activities associated with any of the clinical trials to identify any patients of the medical providers who may be eligible to participate in the clinical trials. The medical providers can also use the clinical trial matching platform to refer any identified qualifying patients to participate in the clinical trials. The clinical trial matching platform can provide information about qualifying patients to trial sponsors of the clinical trials. The clinical trial matching platform can additionally provide navigation services to the patients, for instance to help resolve any issues that might otherwise prevent the patients from participating in the clinical trials.

### RELATED APPLICATIONS

This U.S. patent application claims priority to provisional U.S. Patent Application No. 63/442,049, entitled “CLINICAL TRIAL MATCHING PLATFORM,” filed on Jan. 30, 2023, the entirety of which is incorporated herein by reference.

### BACKGROUND

Clinical trials can be conducted to collect information about the performance of new medications, medical devices, medical treatments, and other interventions that may address medical conditions. Clinical trials can be performed by trial sponsors that want to evaluate the safety and effectiveness of such interventions. Patients may choose to participate in clinical trials to gain access to new interventions that may help treat their medical conditions.

For example, a clinical trial may involve a trial sponsor providing a new medication to a set of participants. Based on data collected during the clinical trial, the trial sponsor can evaluate the safety and effectiveness of the new medication, for instance based on health results of participants who received the new medication relative to health results of other participants who were not given the new medication.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 shows an example of a clinical trial matching platform.

FIG. 2 shows an example of a list of clinical trials that can be displayed via a user interface of the clinical trial matching platform.

FIG. 3 shows an example of a dashboard view that can be displayed via the user interface.

FIG. 4 shows a flowchart illustrating an example method for pre-screening and referring patients for a clinical trial via the clinical trial matching platform.

FIG. 5 shows an example system architecture for a computing system that can execute the clinical trial matching platform.

### DETAILED DESCRIPTION

Clinical trials can be used to evaluate the safety and effectiveness of new medications, medical devices, medical treatments, and other interventions that may address medical conditions. For example, a clinical trial may involve providing a new medication to participants of the clinical trial, and evaluating the safety and effectiveness of the new medication based on reactions of the participants to the new medication relative to other participants who were not given the new medication.

Clinical trials can be sponsored by trial sponsors, such as pharmaceutical companies, governmental agencies, academic institutions, research groups, and/or other entities. Clinical trials may be performed at hospitals, academic institutions, and/or other locations.

It can be difficult for trial sponsors to find participants for clinical trials. For example, a trial sponsor may want to perform a clinical trial for a new medical treatment designed to treat a particular medical condition, but may have difficulty finding people who have that particular medical condition and who will agree to participate in the particular medical condition. Additionally, even if a person is eligible for a clinical trial and may want to participate in the clinical trial, there may be personal and/or logistical issues, such as lodging concerns, transportation concerns, and food insecurity concerns, that may prevent the person from participating in the clinical trial.

It can also be difficult for medical providers to learn about clinical trials that are recruiting participants and that may potentially benefit patients of the medical providers. For example, many medical providers have historically learned about clinical trials through word of mouth, physical corkboard postings, or other conventional methods. While medical providers may learn about a limited set of clinical trials through such conventional methods, such conventional methods may not allow the medical providers to become aware of a full set of clinical trials that may be available to their patients.

Additionally, in conventional systems, a medical provider may suggest that a patient participate in a clinical trial, but the medical provider may have limited abilities to help the patient actually enroll in the clinical trial. As an example, the patient may be responsible for contacting a trial sponsor and/or signing up for the clinical trial. As another example, the trial sponsor of the clinical trial may have to expend time and resources to verify that the patient is eligible to participate in the clinical trial, communicate with the patient to confirm that the patient can participate in the clinical trial, and perform other tasks.

The systems and methods described herein can allow medical providers to view a listing of clinical trials that are recruiting participants. The medical providers can select clinical trials and agree to perform corresponding pre-screening activities that may identify which of their patients are eligible to be participants of the clinical trials. The medical providers can discuss the clinical trials with the identified patients. If the patients agree to participate in the clinical trials, the medical providers can use the systems and methods described herein to refer the patients for the clinical trials, and/or to provide documentation of the eligibility of the patients for the clinical trials. The systems and methods described herein can also provide navigation services for the patients that can resolve transportation issues, lodging issues, food issues, and/or other issues that may otherwise prevent the patients from participating in the clinical trials, so that the patients can successfully participate in the clinical trials.

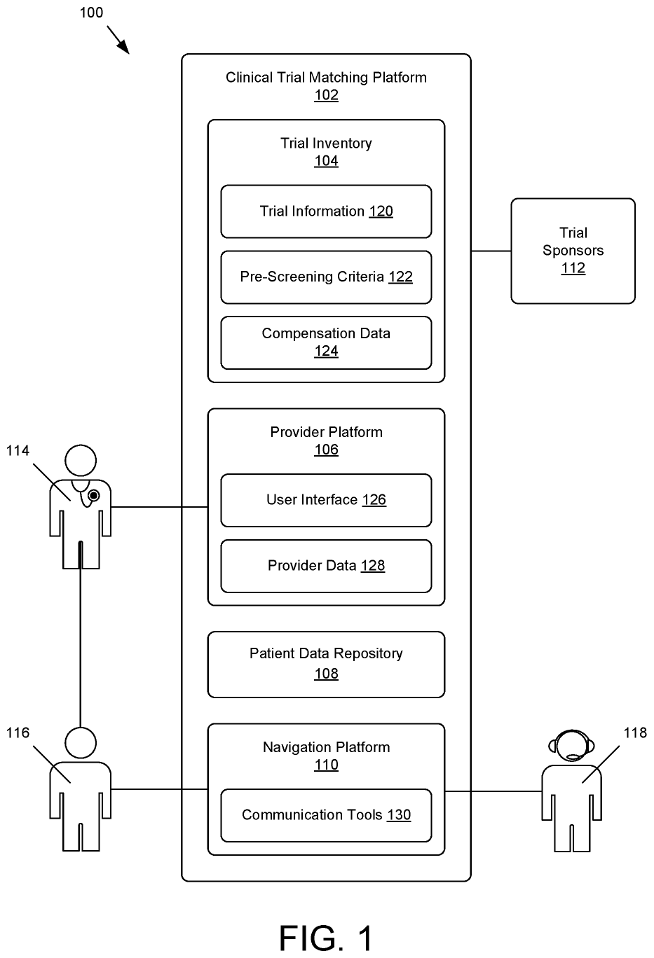

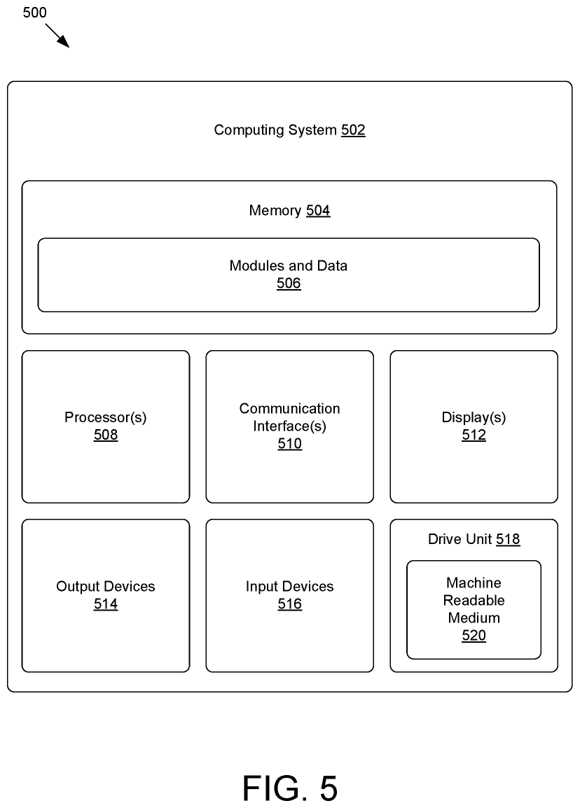

FIG. 1 shows an example 100 of a clinical trial matching platform 102. The clinical trial matching platform 102 can comprise, and/or be provided via, one or more web applications, mobile applications, and/or other executable applications that are executed by one or more servers, user devices, or other computing systems, as discussed further below with respect to FIG. 5. For instance, in some examples, elements of the clinical trial matching platform 102 can be one or more web applications that are executed by servers in a cloud computing environment or by other remote servers. Such web applications may be accessible via web browsers executing on user devices, for instance through one or more websites associated with the clinical trial matching platform 102. The clinical trial matching platform 102 can include a trial inventory 104, a provider platform 106, a patient data repository 108, and/or a navigation platform 110.

As discussed above, clinical trials can be used to evaluate the safety and effectiveness of new medications, medical devices, medical treatments, and other interventions that may address medical conditions. Such clinical trials can be funded, organized, and/or executed by trial sponsors 112, such as pharmaceutical companies, governmental agencies, academic institutions, research groups, and/or other entities. Trial sponsors 112 and/or associated entities may look for participants who agree to participate in clinical trials, for instance when the clinical trials are in a recruitment phase. The clinical trial matching platform 102 can be used to identify clinical trials that are looking for participants, to identify and pre-screen patients who may be eligible to be participants of the clinical trials, and to help the patients participate in the clinical trials.

The provider platform 106 can be a web application, mobile application, or other platform that medical providers 114 can use to search for and/or learn about clinical trials listed in the trial inventory 104. As used herein, the term “medical provider” may refer to a physician, a medical practice or group, a staff member associated with a medical practice or group, and/or any other user or entity associated with a medical provider 114. A medical provider 114 can pre-screen patients 116 of the medical provider 114 to determine whether any of the medical provider's patients 116 may be eligible to participate in any of the clinical trials listed in the trial inventory 104.

If the medical provider 114 determines that a patient 116 of the medical provider 114 is eligible for a clinical trial, the medical provider 114 can discuss the clinical trial with the patient 116. For example, the medical provider 114 may speak with the patient 116 to explain the clinical trial and potential benefits of the patient 116 participating in the clinical trial. If the patient 116 agrees to participate in the clinical trial, patient data associated with the patient 116 can be provided to, and/or stored in, the patient data repository 108 of the clinical trial matching platform 102.

Additionally, or alternately, if the patient 116 agrees to participate in the clinical trial, the patient 116 can communicate with a navigator 118 associated with the clinical trial matching platform 102 via the navigation platform 110. The navigator 118 can assist the patient 116 with transportation issues, lodging issues, food issues, and/or other issues in order to help the patient 116 participate in the clinical trial.

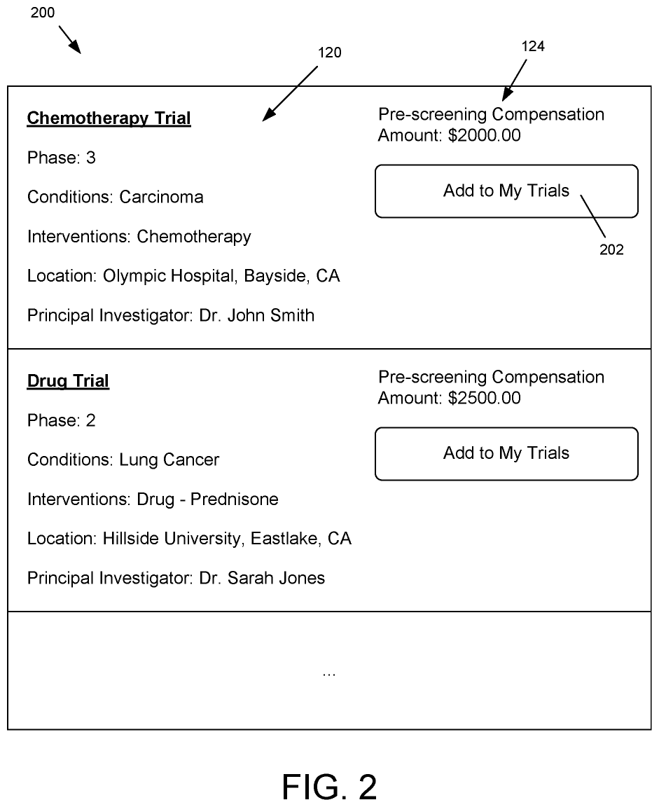

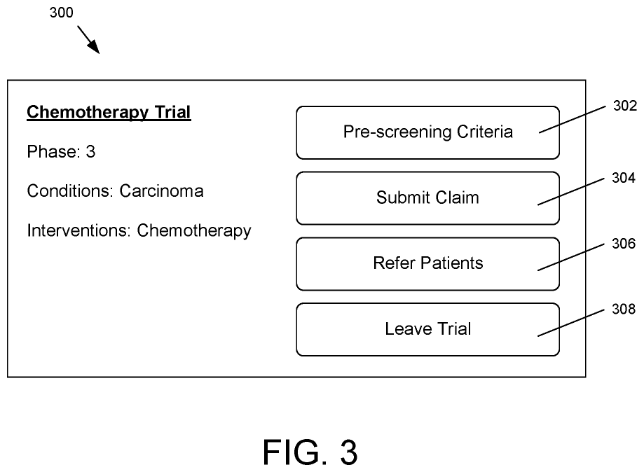

The trial inventory 104 can be a database or other repository of data associated with clinical trials. The trial inventory 104 can store trial information 120, pre-screening criteria 122, and/or compensation data 124 associated with clinical trials. The provider platform 106 can have a user interface 126 that allows a user, such as a medical provider 114 or a staff member associated with a medical provider 114, to view listings of trial information 120, pre-screening criteria 122, and compensation data 124 stored in the trial inventory 104, for example as shown in FIGS. 2 and 3.

The trial information 120 can indicate details of clinical trials. For instance, the trial information 120 can indicate descriptions of clinical trials, conditions treated by the clinical trials, interventions being used and/or studied by the clinical trials, titles of clinical trials, locations of clinical trials, trial sponsors 112 of clinical trials, principal investigators of the clinical trials, and/or any other information about clinical trials.

The pre-screening criteria 122 can indicate desired attributes of participants of the clinical trials. For example, if a patient 116 meets the pre-screening criteria 122 for a clinical trial, the patient 116 may be eligible to be a participant of the clinical trial. The pre-screening criteria 122 may indicate age ranges, genders, medical conditions, demographic attributes, and/or other desired attributes of participants of clinical trials. In some examples, the pre-screening criteria 122 may indicate inclusion criteria and/or exclusion criteria. For instance, inclusion criteria may indicate that a participant should have one or more types of medical conditions to be eligible for a clinical trial, while exclusion criteria may indicate that a participant should not have one or more other types of medical conditions to be eligible for the clinical trial.

The compensation data 124 can indicate a reimbursement or compensation amount that a medical provider 114 can receive, for instance from an operator of the clinical trial matching platform 102, based on the medical provider 114 taking action to pre-screen patients 116 of the medical provider 114 for a corresponding clinical trial. For example, as described further below, a medical provider 114 may agree via the user interface 126 to perform pre-screening activities associated with a particular clinical trial, in exchange for a compensation amount indicated by the compensation data 124.

To pre-screen patients 116 in association with a particular clinical trial identified via the clinical trial matching platform 102, a medical provider 114 can review medical records of the medical provider's patients 116 to determine which of those patients 116, if any, may be eligible to be participants of the particular clinical trial. The medical provider 114 can provide, via the user interface 126, an indication of how many of the medical provider's patients 116 were identified that meet the pre-screening criteria 122 for the particular clinical trial, and may thus be eligible to be participants of the particular clinical trial.

The medical provider 114 can also agree, via the user interface 126, to discuss the particular clinical trial with the identified patients 116 that meet the pre-screening criteria 122. The operator of the clinical trial matching platform 102 can pay, or otherwise transfer, the compensation amount associated with the particular clinical trial to the medical provider 114, based on the medical provider 114 completing the pre-screening activities associated with the particular clinical trial. The medical provider 114 may receive the compensation amount based on completion of the pre-screening activities, regardless of whether any of the patients 116 identified via the pre-screening activities ultimately agree to participate, or do participate, in the clinical trial.

As discussed above, a medical provider 114 can be a physician, a medical practice or group, a staff member associated with a medical practice or group, and/or any other user. In some examples, a particular medical provider 114 can be associated with multiple users of the clinical trial matching platform 102. For instance, a doctor, a nurse, and a staff member associated with the same medical practice may register for separate user accounts with the clinical trial matching platform 102, but may be considered herein to be the same medical provider 114. For example, a doctor may use the provider platform 106 to identify clinical trials that the doctor believes may be of use to his or her patients 116, and can assign a staff member to review medical records of the doctor's patients 116 to identify which of those patients 116 may meet the pre-screening criteria 122 associated with one or more clinical trials. In this example, the doctor and the staff member may be considered herein to be the same medical provider 114, even if the doctor and the staff member have different user accounts with the clinical trial matching platform 102. In other examples, a particular medical provider 114 can be an individual user of the clinical trial matching platform 102.

The provider platform 106 can store provider data 128 associated with individual medical providers 114. The provider data 128 associated with a particular medical provider 114 can indicate a name of the medical provider 114, a location of the medical provider 114, identities of one or more users associated with the medical provider 114, and/or financial account information associated with the medical provider 114 that can be used to pay or transfer compensation amounts. In some examples, provider data 128 associated with a particular medical provider 114 can also indicate other information, such as identities of clinical trials for which the medical provider 114 has agreed to perform pre-screening activities, identities of clinical trials for which the medical provider 114 has completed pre-screening activities, lists of pending and/or completed tasks associated with clinical trials, identities of patients 116 of the medical provider 114 that have agreed to participate in clinical trials, and/or any other information.

In some examples, the provider data 128 associated with a particular medical provider 114 can also indicate preferences that the medical provider 114 has provided to the clinical trial matching platform 102. For example, a medical provider 114 may provide user input indicating that the medical provider 114 is interested in clinical trials related to oncology, or is interested in clinical trials related to one or more types of treatment, and is not interested in other types of clinical trials. Accordingly, the provider platform 106 may be configured to filter information presented via the user interface 126 to the medical provider 114 based on the preferences indicated by the provider data 128.

If a medical provider 114 performs pre-screening activities associated with a clinical trial based on corresponding pre-screening criteria 122, the medical provider 114 can discuss the clinical trial with any patients 116 of the medical provider 114 that meet the pre-screening criteria 122, as discussed above. If one of these patients 116 wants to participate in the clinical trial, the medical provider 114 can provide corresponding patient information, documentation, and/or other data associated with the patient 116 to the clinical trial matching platform 102 via the provider platform 106. As an example, the medical provider 114 may provide or upload patient information, documentation, and/or other data associated with the patient 116 to the clinical trial matching platform 102 via a network. In some examples, the medical provider 114 may also, or alternately, refer the patient 116 to the operator of the clinical trial matching platform 102, such that the patient 116 may provide data to the operator of the clinical trial matching platform 102 directly.

Data associated with the patient 116, provided by the medical provider 114 and/or the patient 116, can be stored in the patient data repository 108. The patient data repository 108 can be a database or other repository of data associated with participants, and/or potential participants, of clinical trials. In some examples, the patient data repository 108 may be a secure and HIPAA-compliant database of patient information.

For example, if a medical provider 114 determines via pre-screening activities that a patient 116 may be eligible to participate in a clinical trial, the medical provider 114 can discuss the clinical trial with the patient 116, and the patient 116 may agree to participate in the clinical trial. The medical provider 114 can use the provider platform 106 to refer the patient 116 as a potential participant of the clinical trial, for instance by entering the patient's name, birthdate, and/or other information via the provider platform 106, uploading medical records and/or other documentation showing eligibility of the patient 116 for the clinical trial, and/or by providing any other data associated with the patient 116 to the clinical trial matching platform 102. The provided data can be stored in the patient data repository 108.

In some examples, patient data associated with a referred patient 116 in the patient data repository 108 can be shared with a trial sponsor 112 of the clinical trial. Accordingly, the trial sponsor 112 may use the patient data to verify that the patient 116 is eligible for the clinical trial, and/or to communicate with the patient 116 to register the patient 116 as a participant of the clinical trial.

Patient data associated with a referred patient 116 in the patient data repository 108 may also, or alternately, be accessible by a navigator 118 or other worker associated with the clinical trial matching platform 102. For example, the navigator 118 or other worker may review provided patient data associated with a referred patient 116 to determine whether all requested data has been provided by the medical provider 114 and/or to verify that the patient 116 is eligible for the clinical trial, for instance before such patient data is shared with a trial sponsor 112. As an example, if the medical provider 114 provides incomplete information associated with a patient 116, a navigator 118 or other worker can contact the medical provider 114 and/or the patient 116 to request additional information about the patient 116. In other examples, the user interface 126 can display notifications of incomplete information to a medical provider 114, for instance by indicating that tasks associated with providing patient data for a clinical trial are not yet complete.

When a medical provider 114 refers a patient 116 for a clinical trial via the clinical trial matching platform 102, the medical provider 114 can indicate whether navigation services are being requested for the patient 116. Navigation services can be provided to the patient 116 via the clinical trial matching platform 102, for instance to identify and address any issues that might otherwise prevent the patient 116 from participating in the clinical trial.

If navigation services are requested for a referred patient 116, a navigator 118 associated with the clinical trial matching platform 102 can be assigned to the patient 116. The navigation platform 110 can have a user interface or other interface that allows the navigator 118 to access patient data, associated with the patient 116, in the patient data repository 108. For example, the navigator 118 can use the navigation platform 110 to determine a phone number or other contact information of the patient 116, review medical records or other documentation that may impact whether the patient 116 can participate in the clinical trial, determine which clinical trial the patient 116 has been referred to participate in, and/or any other tasks.

The navigator 118 can communicate with the patient 116, for instance via one or more communication tools 130. The communication tools 130 can include telephone calls, video conferencing, text messages, email communications, messages sent via the navigation platform 110, and/or other communication tools.

In some examples, the communication tools 130 can be a part of the navigation platform 110. The navigation platform 110 can be a web application, mobile application, or other platform that can be accessed by the patient 116 and/or the navigator 118, and that can allow the patient 116 and/or the navigator 118 to communicate via communication tools 130 incorporated into the web application, mobile application, or other platform. For instance, the navigation platform 110 can have a messaging feature that allows the patient 116 and the navigator 118 to write and send messages to each other, a chat feature that allows the patient 116 and the navigator 118 to engage in text chat sessions, a voice call feature that allows the patient 116 and the navigator 118 to communicate audibly, a videoconference feature that allows the patient 116 and the navigator 118 to communicate via videoconference, and/or any other communication features.

In other examples, the patient 116 and the navigator 118 can communicate via one or more communication tools 130 that are outside the navigation platform 110. For example, the patient 116 and the navigator 118 can communicate via text messages, telephone calls, video chat, and/or other communication tools 130 provided by telecommunication networks or other service providers.

The navigator 118 can provide navigation services to the patient 116. For example, the navigator 118 can set up an initial call, videoconference, or other communication session with the patient 116 to answer any questions the patient 116 may have about the clinical trial. During such an initial communication session, the navigator 118 can also determine if the patient 116 is worried about lodging issues, food issues, transportation issues, or any other issues that may impact the ability of the patient 116 to travel to, and/or stay at, the location of the clinical trial during the patient's participation in the clinical trial. For example, the navigator 118 can evaluate social determinants of health (SDoH) barriers that may prevent the patient 116 from participating in the clinical trial, can counsel the patient 116 about such SDoH barriers, and/or can assist the patient 116 with resolving the SDoH barriers.

In some examples, the navigator 118 and/or the navigation platform 110 can, prior to the initial communication session, provide a survey to the patient 116 about potential issues that may impact the ability of the patient 116 to participate in the clinical trial. The navigation platform 110 may also provide the patient's answers to the survey to the navigator 118. Accordingly, the navigator 118 may review the patient's answers to the survey in advance of the initial communication session, for instance to determine what types of issues the patient 116 may need help with, to research possible solutions, and/or to otherwise prepare for the initial communication session.

After using a survey and/or an initial communication session to determine any issues the patient 116 may need help with, the navigator 118 may set up lodging, food services, transportation, and/or other services for the patient 116. In some examples, the navigator 118 may communicate with one or more partners of the clinical trial matching platform 102 and/or the trial sponsor 112 of the clinical trial to set up such services for the patient 116. The navigator 118 may set up a follow-up communication session with the patient 116, or send information to the patient 116 via the navigation platform 110 or other communication tools 130, to explain details of services that have been set up for the patient 116.

In some examples, the navigator 118 can request that an interpreter join an initial communication session and/or follow-up communication sessions with the patient 116, or use automated translation services during such communication sessions. Accordingly, the navigator 118 can provide navigation services to the patient 116 even if the patient 116 and the navigator 118 speak different languages.

As described above, a medical provider 114 can agree to perform pre-screening activities associated with a particular clinical trial in exchange for payment of a compensation amount. In some examples, such pre-screening activities can be performed by the medical provider 114. For instance, the medical provider 114 may perform pre-screening activities to identify patients 116 of the medical provider 114 that may be eligible to participate in the particular clinical trial, as discussed above.

In other examples, the medical provider 114 can grant the clinical trial matching platform 102 access to medical records of the medical provider 114, such that the clinical trial matching platform 102 can perform the pre-screening activities on behalf of the medical provider 114. As an example, one or more navigators 118 or other workers associated with the clinical trial matching platform 102 can review the medical records of the medical provider 114 to determine whether any patients 116 of the medical provider 114 meet the pre-screening criteria of a clinical trial. As another example, the clinical trial matching platform 102 can use automated document evaluation systems that may use optical character recognition (OCR), natural language processing (NLP), supervised and/or unsupervised machine learning systems, artificial intelligence, and/or other systems or technologies to automatically evaluate the medical records, and to determine whether any patients 116 of the medical provider 114 meet the pre-screening criteria of a clinical trial.

In some examples in which the clinical trial matching platform 102 is granted access to medical records of a medical provider 114 and performs pre-screening activities on behalf of the medical provider 114, the medical records provided by the medical provider 114 can be anonymized. For example, the medical records provided to the clinical trial matching platform 102 can be scrubbed of personal identifiable information of the patients 116. Accordingly, although the clinical trial matching platform 102 may determine that certain medical records indicate that a corresponding patient 116 may be eligible to participate in a clinical trial, the clinical trial matching platform 102 may not have information indicating the identity of that patient 116. The clinical trial matching platform 102 may instead provide a medical record number or some other anonymized identifier of the patient 116 to the medical provider 114, such that the medical provider 114 can determine the identity of that patient 116 and discuss the clinical trial with the patient 116. If the patient 116 then agrees to participate in the clinical trial, the medical provider 114 can provide corresponding patient information to the patient data repository 108 and/or refer the patient 116 to the operator of the clinical trial matching platform 102 as discussed above.

In still other examples, other partners of the clinical trial matching platform 102, such as pharmacies, genomic research entities, benefit plans, insurance providers, and/or other partners, may perform pre-screening activities in association with clinical trials to identify patients 116 that may be eligible for the clinical trials. In some of these examples, the partners or the clinical trial matching platform 102 can use automated systems and/or artificial intelligence techniques to automatically evaluate data stored by, or available to, the partners to determine whether any corresponding patients 116 may be likely to meet the pre-screening criteria of a clinical trial. Such automated systems and/or artificial intelligence techniques can include OCR, NLP, supervised machine learning, unsupervised machine learning, and/or other artificial intelligence techniques. If a partner of the clinical trial matching platform 102 identifies any patients patient 116 that may be eligible for clinical trials, the partners may notify the clinical trial matching platform 102. The clinical trial matching platform 102 can in turn contact medical providers 114 associated with those patients 116, so that the medical providers 114 can discuss the clinical trials with the patients 116 and/or refer the patients 116 for the clinical trials.

As an example, an insurance company may partner with the clinical trial matching platform 102, and may use records available to the insurance company to determine that a particular patient 116 may be eligible for a particular clinical trial. The insurance company may inform the clinical trial matching platform 102 that the patient 116 may be eligible for the clinical trial. The clinical trial matching platform 102 can notify a medical provider 114 associated with the patient 116, such as the patient's primary care doctor, about the clinical trial. The medical provider 114 can register with the clinical trial matching platform 102 if the medical provider 114 has not yet done so, can review pre-screening criteria 122 associated with the clinical trial via the user interface 126, can determine whether the patient 116 meets the pre-screening criteria 122, and/or can discuss the clinical trial with the patient 116 as discussed above. If the patient 116 agrees to participate in the clinical trial, the medical provider 114 can refer the patient 116 for the clinical trial via the clinical trial matching platform 102 as discussed above, for instance by providing additional medical records or documentation to confirm that the patient 116 is eligible to be a participant in the clinical trial.

The clinical trial matching platform 102 may be operated by an entity that is different from the trial sponsors 112. In some examples, trial sponsors 112 may pay referral fees to the operator of the clinical trial matching platform 102 for each patient 116 that is referred for clinical trials via the clinical trial matching platform 102. However, the referral fees paid by the trial sponsors 112 to the operator of the clinical trial matching platform 102 for each referred patient can be independent from compensation amounts that the operator of the clinical trial matching platform 102 pays to medical providers 114 in exchange for the medical providers 114 performing pre-screening activities for clinical trials.

In some examples, the operator of the clinical trial matching platform 102 can communicate with trial sponsors 112 to determine target demographics for clinical trials, locations of clinical trials, pre-screening criteria 122 associated with the clinical trials, and/or other trial information 120 associated with the clinical trials. In these examples, the clinical trial matching platform 102 can display information about the clinical trials to medical providers 114 via the user interface 126 based on communications and/or agreements with the trial sponsors 112. In other examples, the operator of the clinical trial matching platform 102 may learn about clinical trials through journals, online postings, and/or other sources, and may display information about the clinical trials to medical providers 114 via the user interface 126.

In some examples, the clinical trial matching platform 102 can be configured to present different sets of clinical trials to different medical providers 114 via the user interface 126, based on target demographics for clinical trials, demographics of areas served by the different medical providers 114, based on distances between locations of the medical providers 114 and clinical trials, and/or any other information. As an example, a trial sponsor 112 may indicate that more Hispanic participants are desired for a particular clinical trial that will be performed at a particular location. Accordingly, based on provider data 128 indicating locations of different medical providers 114, and demographic information about populations that live in or around those locations, the clinical trial matching platform 102 can identify a set of medical providers 114 that are located in areas with large Hispanic populations and that are within a threshold distance away from the location of the particular clinical trial. The clinical trial matching platform 102 can thus list the particular clinical trial via the user interface 126 to that set of medical providers 114, but may avoid listing the particular clinical trial via the user interface 126 to other medical providers 114 that are in areas with smaller Hispanic populations and/or that are more than the threshold distance away from the location of the clinical trial. By listing the particular clinical trials to a particular set of medical provider 114 that are located in areas with large Hispanic populations, the clinical trial matching platform 102 may increase the chances that more Hispanic participants are found for the particular clinical trial.

In other examples, the user interface 126 of the provider platform 106 can display information about sets of clinical trials that may be of interest to a medical provider 114 based on preferences of the medical provider 114 stored in the provider data 128, and/or based on search criteria entered by the medical provider 114. For example, the user interface 126 can have a search feature that allows a medical provider 114 to search for clinical trials that will be performed at a location entered by the medical provider 114 or within threshold distances of that location, that are intended to treat medical conditions input by the medical provider 114, that will use types of interventions input by the medical provider 114, that are being performed by trial sponsors 112 or categories of trial sponsors 112 entered by the medical provider 114, and/or that otherwise meet search criteria entered by the medical provider 114. In some examples, the user interface 126 may display information about clinical trials for which the operator of the clinical trial matching platform 102 is offering compensation amounts for pre-screening activities, and/or for which compensation amounts for pre-screening activities are not being offered.

The user interface 126 may also allow a medical provider 114 to exchange messages with trial sponsors 112 or associated personnel. For example, if a medical provider 114 has a question about a clinical trial, the user interface 126 may have communication tools that allow the medical provider 114 to send a message to the principal investigator of the clinical trial, and for the principal investigator to send a response back to the medical provider 114.

The clinical trial matching platform 102 can provide updates about a patient's referral to a clinical trial to the patient 116, the patient's medical provider 114, and/or a corresponding trial sponsor 112, for instance to indicate when communications between the patient 116 and a navigator 118 have occurred, when medical reports or other documentation have been requested and/or received, when and where the patient is to participate in the clinical trial, and/or any other information. In some examples, the clinical trial matching platform 102 can also provide updates to patients 116, medical providers 114, and/or trial sponsors 112 about the clinical trials. For example, the clinical trial matching platform 102 can track a patient's progress through a clinical trial, and provide updates to the patient's medical provider.

In some examples, when patients 116 are referred via the clinical trial matching platform 102 for clinical trials, the clinical trial matching platform 102 can verify that the patients 116 are eligible for the clinical trials, and can provide reports about the patients 116 to the corresponding trial sponsors 112. Such reports can reduce the burden on the trial sponsors 112 by reducing the risks of non-qualified patients, and/or patients who may not be able to actually participate in the clinical trials due to SDoH barriers, being referred to the clinical trials. For instance, navigators 118 may communicate with patients 116, referring medical providers 114, and/or trial sponsors 112 to verify that referred patients 116 meet pre-screening criteria 122 of clinical trials, and can help resolve any SDoH barriers that the patients 116 may have. The clinical trial matching platform 102 can also provide corresponding pre-screening data, medical records, SDoH assessments, and/or other information to the trial sponsors 112, such that the trial sponsors 112 can avoid spending time, computing resources, and other resources obtaining and/or verifying that information.

Accordingly, the clinical trial matching platform 102 can provide benefits over conventional systems associated with clinical trials. For example, conventionally a trial sponsor 112 may use time, processing resources, bandwidth resources, other computing resources, and/or other resources to contact a potential participant of a clinical trial, and/or to obtain and verify information associated with the potential participant. However, the trial sponsors 112 may ultimately determine that the potential participant does not actually qualify to be a participant of the clinical trial, or the potential participant may have concerns or experience issues that prevent the potential participant from participating in the clinical trial. In such situations, resources that were used by the trial sponsors 112 to contact the potential participant, and/or to evaluate information associated with the potential participant, may be wasted. However, as described herein, the clinical trial matching platform 102 may provide a trial sponsor 112 with information about patient 116 who have already been pre-screened and vetted for the clinical trial by medical providers 114 and/or the clinical trial matching platform 102, and/or who are being assisted by navigators 118 to resolve issues that may otherwise prevent participation in the clinical trial. Accordingly, patients 116 referred to trial sponsors 112 via the clinical trial matching platform 102 may be more likely to become actual participants of corresponding clinical trials, and thereby reduce the time and amounts of processing resources, bandwidth resources, other computing resources, and/or other resources used by the trial sponsors 112.

FIG. 2 shows an example 200 of a list of clinical trials that can be displayed via the user interface 126 of the provider platform 106. The user interface 126 can display information about clinical trials that are recruiting participants, for instance by presenting corresponding trial information 120 such as titles of the clinical trials, conditions treated by the clinical trials, interventions being used and/or studied in the clinical trials, principal investigators of the clinical trials, and/or other information about the clinical trials.

The user interface 126 can also display compensation data 124 associated with individual clinical trials. As discussed above, the compensation data 124 can indicate reimbursement or compensation amounts that an operator of the clinical trial matching platform 102 will pay medical providers 114 for performing pre-screening activities associated with the clinical trials. As a non-limiting example, in FIG. 2 the user interface 126 indicates that a medical provider would receive $2000.00 for performing pre-screening activities associated with a chemotherapy trial, and would receive $2500.00 for performing pre-screening activities associated with a chemotherapy trial. In other examples, the reimbursement or compensation amounts associated with clinical trials can have other values.

As discussed above, the provider platform 106 can be configured to present different lists of clinical trials to different medical providers 114 via the user interface 126. For example, if a particular clinical trial is looking for participants in a particular area and/or that have target demographic attributes, the user interface 126 may display that particular clinical trial to medical providers 114 that are within a threshold distance of that particular area and/or that serve areas with populations that correspond with the target demographic attributes. However, the user interface 126 may display the particular clinical trial to other medical providers 114 that are farther away from the particular area, or that serve populations that do not correspond with the target demographic attributes. As another example, the provider platform 106 can filter clinical trials in the trial inventory 104 based on provider preferences indicated in the provider data 128, and/or based on search terms entered by the medical providers 114, such that the user interface 126 displays a filtered list of clinical trials that may be of the most interest to the medical providers 114.

A medical provider 114 can review the list of clinical trials displayed via the user interface 126. The medical provider 114 can also determine whether to select any of the clinical trials based on the corresponding trial information 120, compensation data 124, and/or other information. If the medical provider 114 chooses to select any of the listed clinical trials, the medical provider 114 can select an “add to my trials” button 202 or other user interface element. The selected clinical trial can be added to a dashboard of selected clinical trials in the user interface 126, such that the medical provider 114 can perform one or more tasks associated with the clinical trials as discussed below with respect to FIG. 3.

FIG. 3 shows an example 300 of a dashboard view that can be displayed via the user interface 126, which can present user-selectable elements associated with tasks that correspond to one or more clinical trials that have been selected by a medical provider 114. For example, if a medical provider 114 uses an “add to my trials” button 202 as shown in FIG. 2 to select a clinical trial, the clinical trial can be added to the dashboard view shown in FIG. 3. The dashboard view can display user-selectable elements associated with the clinical trial, including a “pre-screening criteria” button 302, a “submit claim” button 304, a “refer patients” button 306, a “leave trial” button 308, and/or other user-selectable elements.

The medical provider 114 can select the “pre-screening criteria” button 302 to view the pre-screening criteria 122 associated with the clinical trial. The medical provider 114 can choose to perform pre-screening activities by reviewing medical records and/or otherwise screening patients 116 of the medical provider 114 to determine which patients 116 of the medical provider 114 may be eligible for the clinical trial.

When the medical provider 114 has completed the pre-screening activities based on the pre-screening criteria 122, the medical provider 114 can use the “submit claim” button 304 to apply for reimbursement or compensation for performing the pre-screening activities. For example, selection of the “submit claim” button 304 can prompt the user interface 126 to display one or more other windows, tabs, or other views that allow the medical provider 114 to verity that the pre-screening activities have been completed, and to input a number of patients 116 of the medical provider 114 that the medical provider 114 believes are eligible for the clinical trial based on the pre-screening activities. The user interface 126 may also display a notification indicating that the medical provider 114 should additionally pre-screen any new or existing patients that are seen by the medical provider 114 during future visits over the next month, three months, or any other future period of time. The user interface 126 may additionally request user input indicating how the pre-screening activities were performed, indicating which users or personnel associated with the medical provider 114 performed the pre-screening activities, indicating the time it took to perform the pre-screening activities, and/or indicating any other details about the pre-screening activities performed by the medical provider 114. The user interface 126 can additionally display messages asking the medical provider 114 to agree to discuss the clinical trial with the patients 116 identified during the pre-screening activities, and/or to enter a date of when the medical provider 114 believes such discussions with the identified patients 116 will be complete.

Once the medical provider 114 has selected the “submit claim” button 304 and has entered details about the pre-screening activities performed by the medical provider 114 in association with a clinical trial, the medical provider 114 can submit a corresponding reimbursement or compensation claim for performing the pre-screening activities. For example, if the medical provider 114 submits a claim associated pre-screening activities for a clinical trial, for instance by indicating how many qualifying patients 116 were identified, by agreeing to discuss the clinical trial with the identified qualifying patients 116, and by providing an approximate date of when discussions of the clinical trial with the identified qualifying patients 116 will be complete, the operator of the clinical trial matching platform 102 can pay the medical provider 114 a corresponding compensation amount indicated by the compensation data 124 associated with the clinical trial. As discussed above, the compensation amount can be paid in exchange for the medical provider 114 completing the pre-screening activities, including identifying how many qualifying patients 116 were identified and for agreeing to discuss the clinical trial with those patients 116, and can be paid regardless of whether any of those patients 116 agree to actually participate in the clinical trial.

After the medical provider 114 has completed the pre-screening activities and has submitted a claim for compensation, the medical provider 114 may discuss the clinical trial with patients 116 identified by the pre-screening activities that may qualify for the clinical trial. For example, the medical provider 114 can discuss the benefits and/or risks of the clinical trial with an identified patient 116. If any of the identified patients 116 agree to participate in the clinical trial after discussing the clinical trial with the medical provider 114, the medical provider 114 can initiate a referral of the patient 116 for the clinical trial by selecting the “refer patients” button 306.

For example, selection of the “refer patients” button 306 can allow the medical provider 114 to input details associated with a patient via the user interface 126, such as the patient's name, date of birth, contact information, one or more locations of the clinical trial that are preferred by the patient (for instance if the clinical trial will be held at multiple locations), and/or other patient data. The user interface 126 can also request that the medical provider 114 upload, or otherwise provide access, to medical records or other documentation verifying that the patient 116 meets the pre-screening criteria for the clinical trial. The user interface 126 can also have a selectable option that allows the medical provider 114 to indicate whether navigation services are being requested for the patient 116, which may involve a navigator 118 assisting the patient 116 with one or more issues to help the patient 116 participate in the clinical trial as discussed above.

If the medical provider 114 chooses to not perform pre-screening activities in association with the clinical trial, or otherwise wants to disassociate with the clinical trial, the medical provider 114 can select the “leave trial” button 308. Selection of the “leave trial” button 308 can remove the clinical trial from a dashboard view in the user interface.

In some examples, the dashboard view shown in FIG. 3 can also display other information or notifications associated with the clinical trial. For example, the dashboard view may display information about a pending claim for compensation that has already been submitted, notifications of missing medical records or other documentation that are to be provided for patients 116 referred for the clinical trial, and/or any other information. In other example, the dashboard view or other portions of the user interface 126 may display lists of patients 116 that have been referred for clinical trials, which clinical trials are associated with which patients 116, and/or any other information associated with the medical provider 114 and/or patients of the medical provider 114.

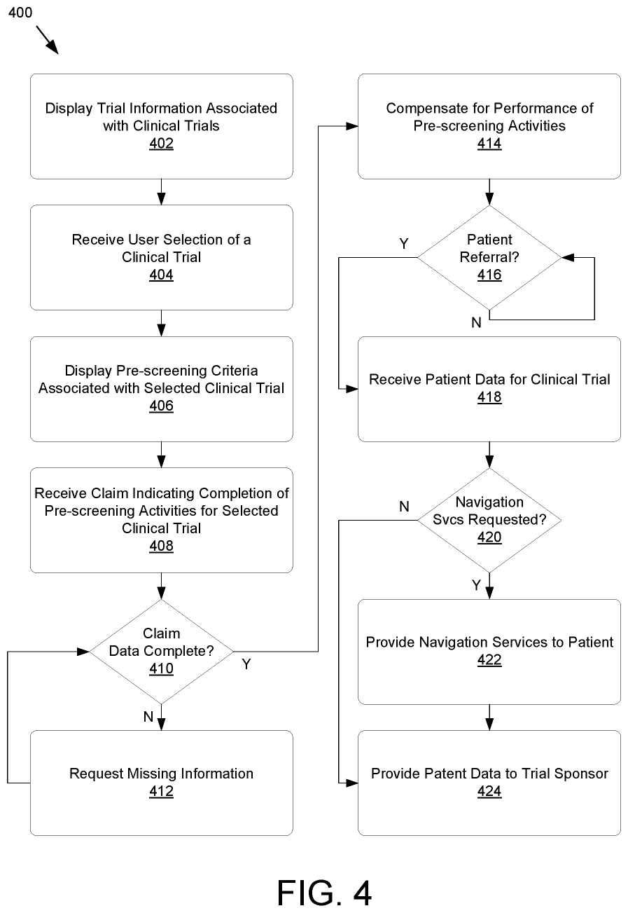

FIG. 4 shows a flowchart illustrating an example method 400 for pre-screening and referring patients for a clinical trial via the clinical trial matching platform 102. The method 400 shown in FIG. 4 can be performed by elements of the clinical trial matching platform 102 executing via one or more computing systems. An example system architecture for such a computing system is described below with respect to FIG. 5.

At block 402, the user interface 126 of the clinical trial matching platform 102 can display trial information 120 associated with one or more clinical trials to a medical provider 114. The clinical trial matching platform 102 may filter or select which clinical trials are presented to the medical provider 114 via the user interface 126 based on target locations, demographics, and/or other attributes specified by trial sponsors 112, and/or based on preferences or search criteria entered by the medical provider 114.

At block 404, the clinical trial matching platform 102 can receive a user selection of a particular clinical trial via the user interface 126. For example, the medical provider 114 may view trial information 120 associated with multiple clinical trials presented at block 402, and may select one of those clinical trials at block 404. At block 406, the clinical trial matching platform 102 can display pre-screening criteria 122 associated with the clinical trial selected at block 404.

At block 408, the clinical trial matching platform 102 can receive a submission of a claim from the medical provider 114 indicating that the medical provider 114 has completed pre-screening activities associated with the pre-screening criteria 122 for the clinical trial. As discussed above, the submitted claim data can indicate a number of patients 116 identified by the medical provider that meet the pre-screening criteria 122, an agreement of the medical provider 114 to discuss the clinical trial with the identified patients, and/or other information about how the medical provider 114 completed the pre-screening activities.

At block 410, the clinical trial matching platform 102 can determine whether the claim data submitted by the medical provider 114 at block 408 is complete. If the claim data submitted by the medical provider 114 is not yet complete (Block 410—No), for instance if required information about how the medical provider 114 performed the pre-screening activities has not yet been received, the clinical trial matching platform 102 can request the missing information from the medical provider 114 at block 412. For instance, the clinical trial matching platform 102 may display a notification via the user interface 126 that informs the medical provider that the claim is not yet complete and/or that identifies the missing information that is still to be received.

If the claim data submitted by the medical provider 114 is complete (Block 410—Yes), for instance if all required information about how the medical provider 114 performed the pre-screening activities has been received, the clinical trial matching platform 102 can compensate the medical provider 114 for performing the pre-screening activities at block 414. For example, the clinical trial matching platform 102 can pay the medical provider 114 a corresponding compensation amount indicated by the compensation data 124 associated with the clinical trial.

At block 416, the clinical trial matching platform 102 can determine whether the medical provider 114 has referred a patient 116 for the clinical trial. For example, after performing the pre-screening activities as discussed above, the medical provider 114 can discuss the clinical trial with any patients 116 of the medical provider 114 that met the pre-screening criteria and that may thus be eligible to participate in the clinical trial. If any of those patients 116 agree to participate in the clinical trial, the medical provider 114 can use the user interface 126 to refer the patient 116 for the clinical trial, for instance by selecting a “refer patient” button 306 as shown in FIG. 3.

If the clinical trial matching platform 102 determines that the medical provider 114 is referring a patient 116 for the clinical trial (Block 416—Yes), the clinical trial matching platform 102 can receive patient data associated with the patient 116 for the clinical trial at block 418. For example, the medical provider 114 can use the provider platform 106 to enter the patient's name, contact information, and/or other details, to upload medical records or other documentation demonstrating that the patient is eligible for the clinical trial, to provide an indication of whether navigation services are being requested for the patient, and/or to provide any other patient information.

At block 420, the clinical trial matching platform 102 can determine whether navigation services have been requested for the patient 116. If navigation services have been requested for the patient 116 (Block 420—Yes), the clinical trial matching platform 102 can provide the requested navigation services to the patient 116 at block 422. For example, the clinical trial matching platform 102 can assign a navigator 118 to assist the patient 116, and the navigator 118 and/or the patient 116 can use the navigation platform 110 and/or communication via the navigation platform 110 so that the navigator 118 can provide navigation services that may assist the patient 116 with participation in the clinical trial.

If navigation services have not been requested for the patient 116 (Block 420—No), and/or if navigation services were requested and have been provided to the patient 116 at block 422, and the patient 116 will be able to participate in the clinical trial, the clinical trial matching platform 102 can provide corresponding patient data to the trial sponsor 112 associated with the clinical trial at block 424. In some examples, the clinical trial matching platform 102 or a navigator 118 may review patient data or other data provided by the medical provider 114 and/or the patient 116 to verify that the patient does meet the pre-screening criteria for the clinical trial, to request any missing or additional information from the medical provider 114 and/or the patient 116, and/or otherwise verify that all relevant patient data has been received by the clinical trial matching platform 102 before the clinical trial matching platform 102 provides the patient data to the trial sponsor 112 at block 424.

The clinical trial matching platform 102 can repeat block 416 through block 424 for multiple patients 116. For example, if the medical provider 114 determines via pre-screening activities that five patients 116 of the medical provider 114 are eligible to participate in a particular clinical trial, and three of those patients 116 agree to participate in the clinical trial, the clinical trial matching platform 102 may repeat blocks 416 through 424 three times to receive referrals of the three patients 116, receive patient data associated with the three patients 116, provide navigation services to zero or more of the three patients 116, and/or to provide patient data associated with the three patients 116 to the trial sponsor 112 of the clinical trial.

Additionally, the clinical trial matching platform 102 can repeat block 404 through block 424 for multiple clinical trials selected by the medical provider 114. For instance, the medical provider 114 may select a first clinical trial at block 404, submit a corresponding compensation claim at block 408 for performing pre-screening activities associated with the first clinical trial, and may refer zero or more patients 116 for the first clinical trial at block 416. However, at another point in time the medical provider 114 may select a second clinical trial at block 404, submit a corresponding compensation claim at block 408 for performing pre-screening activities associated with the second clinical trial, and may refer zero or more patients 116 for the second clinical trial at block 416.

FIG. 5 shows an example system architecture 500 for a computing system 502 that can execute one or more elements of the clinical trial matching platform 102 described herein. For example, the computing system 502 may store and/or execute one or more portions of the clinical trial matching platform 102, such as the trial inventory 104, the provider platform 106, the patient data repository 108, and/or the navigation platform 110. The computing system 502 can include one or more servers, computers, or other types of computing devices. Individual computing devices of the computing system 502 may have the system architecture 500 shown in FIG. 5, or a similar system architecture.

In some examples, elements of the clinical trial matching platform 102 can be distributed among, and/or be executed by, multiple computing systems or devices similar to the computing system 502 shown in FIG. 5. For example, the provider platform 106 may be executed by a different computing system than the navigation platform 110.

The computing system 502 may, in some examples, be part of a cloud computing environment and/or a software as a service (SaaS) environment that hosts and/or executes one or more elements of the clinical trial matching platform 102. For instance, a cloud computing environment may include multiple servers or other computing devices that can host elements of the clinical trial matching platform 102 and/or multiple instances of the clinical trial matching platform 102. In some examples, the servers or other computing devices of such a cloud computing environment may use one or more virtual machines, containers, and/or other systems to execute one or more instances of the clinical trial matching platform 102

Additionally, in some examples, computing systems and devices similar to the computing system 502 shown in FIG. 5 can be used by medical providers 114, patients 116, and/or navigators 118. For example, a medical provider 114 can use a computer, smartphone, or other computing device to access the provider platform 106 as a web application, mobile application, or other platform. Similarly, a patient 116 and/or a navigator 118 can similarly use computers, smartphones, or other computing devices to access the navigation platform 110 and/or other elements of the clinical trial matching platform 102 as a web application, mobile application, or other platform.

The computing system 502 can include memory 504. In various examples, the memory 504 can include system memory, which may be volatile (such as RAM), non-volatile (such as ROM, flash memory or other solid-state memory that may be used in solid-state drives or other forms of system memory, etc.) or some combination of the two. The memory 504 can further include non-transitory computer-readable media, such as volatile and nonvolatile, removable and non-removable media implemented in any method or technology for storage of information, such as computer readable instructions, data structures, program modules, or other data. System memory, removable storage, and non-removable storage are all examples of non-transitory computer-readable media. Examples of non-transitory computer-readable media include, but are not limited to, RAM, ROM, EEPROM, flash memory or other solid-state memory, such as solid-state memory used in system memory or non-removable storage, other memory technology, magnetic cassettes, magnetic tape, magnetic disk storage or other magnetic storage devices, or any other non-transitory medium which can be used to store desired information and which can be accessed by the computing system 502 associated with the clinical trial matching platform 102. Any such non-transitory computer-readable media may be part of the computing system 502.

The memory 504 can store modules and data 506, such as one or more types of data, computer-executable instructions associated software elements, firmware elements, or other executable elements, and/or other information. The modules and data 506 can be associated with one or more of the trial inventory 104, the provider platform 106, the patient data repository 108, and/or the navigation platform 110, and/or other elements associated with the clinical trial matching platform 102 described herein. In some examples, computer readable instructions, program modules, and other data associated with executable components of the clinical trial matching platform 102 can be stored on one or more types of non-transitory computer-readable media as described above, while the patient data repository 108 and/or other patient data can be stored on non-removable storage to protect privacy of the patients 116 and avoid unauthorized removal of patent data from the computing system 502.

Additionally, or alternately, the modules and data 506 can include any other modules and/or data that can be utilized by the computing system 502 to perform or enable performing any action taken by the computing system 502. Such other modules and data can include a platform, operating system, and/or applications, as well as data utilized by the platform, operating system, and/or applications.

The computing system 502 can also have processor(s) 508, communication interfaces 510, a display 512, output devices 514, input devices 516, and/or a drive unit 518 including a machine readable medium 520.

In various examples, the processor(s) 508 can be a central processing unit (CPU), a graphics processing unit (GPU), both a CPU and a GPU, or any other type of processing unit. Each of the one or more processor(s) 508 may have numerous arithmetic logic units (ALUs) that perform arithmetic and logical operations, as well as one or more control units (CUs) that extract instructions and stored content from processor cache memory, and then executes these instructions by calling on the ALUs, as necessary, during program execution. The processor(s) 508 may also be responsible for executing computer applications stored in the memory 504, which can be associated with common types of volatile (RAM) and/or nonvolatile (ROM) memory.

The communication interfaces 510 can include transceivers, modems, interfaces, antennas, telephone connections, and/or other components that can transmit and/or receive data over networks, telephone lines, or other connections.

The display 512 can be a liquid crystal display, or any other type of display commonly used in computing devices. For example, a display 512 may be a touch-sensitive display screen, and can then also act as an input device or keypad, such as for providing a soft-key keyboard, navigation buttons, or any other type of input.

The output devices 514 can include any sort of output devices known in the art, such as the display 512, speakers, a vibrating mechanism, and/or a tactile feedback mechanism. Output devices 514 can also include ports for one or more peripheral devices, such as headphones, peripheral speakers, and/or a peripheral display.

The input devices 516 can include any sort of input devices known in the art. For example, input devices 516 can include a microphone, a keyboard/keypad, and/or a touch-sensitive display, such as the touch-sensitive display screen described above. A keyboard/keypad can be a push button numeric dialing pad, a multi-key keyboard, or one or more other types of keys or buttons, and can also include a joystick-like controller, designated navigation buttons, or any other type of input mechanism.

The machine readable medium 520 can store one or more sets of instructions, such as software or firmware, that embodies any one or more of the methodologies or functions described herein. The instructions can also reside, completely or at least partially, within the memory 504, processor(s) 508, and/or communication interface(s) 510 during execution thereof by the computing system 502. The memory 504 and the processor(s) 508 also can constitute machine readable media 520.

Although the subject matter has been described in language specific to structural features and/or methodological acts, it is to be understood that the subject matter is not necessarily limited to the specific features or acts described above. Rather, the specific features and acts described above are disclosed as example embodiments.

What is claimed is:

1. A method, comprising:
displaying, by a clinical trial matching platform executed by one or more processors of a computing system, and via a user interface, information indicating:
pre-screening criteria associated with corresponding clinical trials, and
compensation data associated with pre-screening activities, based on the pre-screening criteria, associated with the corresponding clinical trials;
receiving, by the clinical trial matching platform, an indication that the pre-screening activities associated with a clinical trial have been completed in association with a provider;
determining, by the clinical trial matching platform, and based on the indication, a number of patients of the provider that are eligible for the clinical trial;
causing, by the clinical trial matching platform, a transfer of an amount to the provider based on completion of the pre-screening activities and the compensation data;
receiving, by the clinical trial matching platform, patient data associated with a patient;
determining, by the clinical trial matching platform, that the patient is eligible for the clinical trial; and
providing, by the clinical trial matching platform, the patient data to a trial sponsor of the clinical trial based on determining that the patient is eligible for the clinical trial.

2. The method of claim 1, wherein the indication is received via the user interface of the clinical trial matching platform as user input from the provider indicating that the provider has performed the pre-screening activities.

3. The method of claim 1, further comprising performing, by the clinical trial matching platform, the pre-screening activities by:
accessing records of the provider; and
automatically evaluating the records to identify whether any corresponding patients are eligible for the clinical trial based on the pre-screening criteria.

4. The method of claim 1, wherein the information displayed via the user interface is associated with clinical trials identified by the clinical trial matching platform based on at least one of:
a location of the provider,
population demographics of areas served by the provider,
target locations for the clinical trials,
target demographics for participants of the clinical trials,
preferences entered by the provider, or
search criteria entered by the provider.

5. The method of claim 1, wherein the patient data is received in association with a referral of the patient for the clinical trial submitted via the clinical trial matching platform.

6. The method of claim 1, wherein:
the patient data indicates a request for navigation services,
the method further comprises initiating, by the clinical trial matching platform, a communication session between the patient and a navigator associated with the clinical trial matching platform, and
the navigator provides the navigation services during or after the communication session to assist the patient in participating in the clinical trial.

7. The method of claim 1, further comprising:
receiving, by the clinical trial matching platform, a notification from a partner indicating that an evaluation of records of the partner, associated with the patient, indicates that the patient is likely to meet the pre-screening criteria for the clinical trial; and
providing, by the clinical trial matching platform, the provider with the information associated with the clinical trial in response to the notification indicating that the patient is likely to meet the pre-screening criteria for the clinical trial.

8. A computing system, comprising:
at least one processor; and
memory storing computer-executable instructions associated with a clinical trial matching platform that, when executed by the at least one processor, cause the at least one processor to perform operations comprising:
displaying, in a user interface, information indicating:
pre-screening criteria associated with individual clinical trials, and
compensation data associated with pre-screening activities, based on the pre-screening criteria, associated with the corresponding clinical trials;
receiving an indication that the pre-screening activities associated with a clinical trial have been completed in association with a provider;
determining, based on the indication, a number of patients of the provider that are eligible for the clinical trial;
causing a transfer of an amount to the provider based on completion of the pre-screening activities and the compensation data;
receiving patient data associated with a patient;
determining that the patient is eligible for the clinical trial; and
providing the patient data to a trial sponsor of the clinical trial based on determining that the patient is eligible for the clinical trial.

9. The computing system of claim 8, wherein the indication is received via the user interface as user input from the provider indicating that the provider has performed the pre-screening activities.

10. The computing system of claim 8, wherein the operations further comprise performing the pre-screening activities by:
accessing records of the provider; and
automatically evaluating the records to identify whether any corresponding patients are eligible for the clinical trial based on the pre-screening criteria.

11. The computing system of claim 8, wherein the information displayed via the user interface is associated with clinical trials is identified based on at least one of:
a location of the provider,
population demographics of areas served by the provider,
target locations for the clinical trials,
target demographics for participants of the clinical trials,
preferences entered by the provider, or
search criteria entered by the provider.

12. The computing system of claim 8, wherein the patient data is received in association with a referral of the patient for the clinical trial submitted via the clinical trial matching platform.

13. The computing system of claim 8, wherein:
the patient data indicates a request for navigation services,
the operations further comprise initiating a communication session between the patient and a navigator associated with the clinical trial matching platform, and
the navigator provides the navigation services during or after the communication session to assist the patient in participating in the clinical trial.

14. The computing system of claim 8, wherein the operations further comprise:
receiving a notification, from a partner of the clinical trial matching platform, indicating that an evaluation of records of the partner, associated with the patient, indicates that the patient is likely to meet the pre-screening criteria for the clinical trial; and
providing, by the clinical trial matching platform, the provider with the information associated with the clinical trial in response to the notification indicating that the patient is likely to meet the pre-screening criteria for the clinical trial.

15. One or more non-transitory computer-readable media storing computer-executable instructions associated with a clinical trial matching platform that, when executed by at least one processor, cause the at least one processor to perform operations comprising:
displaying, in a user interface of the clinical trial matching platform, information indicating:
pre-screening criteria associated with corresponding clinical trials, and
compensation data associated with pre-screening activities, based on the pre-screening criteria, associated with the corresponding clinical trials;
receiving an indication that pre-screening activities associated with a clinical trial have been completed in association with a provider;
determining, based on the indication, a number of patients of the provider that are eligible for the clinical trial;
causing a transfer of an amount to the provider based on completion of the pre-screening activities and the compensation data;
receiving patient data associated with a patient;
determining that the patient is eligible for the clinical trial; and
providing the patient data to a trial sponsor of the clinical trial based on determining that the patient is eligible for the clinical trial.

16. One or more non-transitory computer-readable media of claim 15, wherein the indication is received as user input from the provider indicating that the provider has performed the pre-screening activities.

17. One or more non-transitory computer-readable media of claim 15, wherein the operations further comprise performing the pre-screening activities by:
accessing records of the provider; and
automatically evaluating the records to identify whether any corresponding patients are eligible for the clinical trial based on the pre-screening criteria.

18. The one or more non-transitory computer-readable media of claim 15, wherein the information displayed via the user interface is associated with clinical trials identified based on at least one of:
a location of the provider,
population demographics of areas served by the provider,
target locations for the clinical trials,
target demographics for participants of the clinical trials,
preferences entered by the provider, or
search criteria entered by the provider.

19. The one or more non-transitory computer-readable media of claim 15, wherein the patient data is received in association with a referral of the patient for the clinical trial submitted via the clinical trial matching platform.

20. The one or more non-transitory computer-readable media of claim 15, wherein:
the patient data indicates a request for navigation services;
the operations further comprise initiating a communication session between the patient and a navigator associated with the clinical trial matching platform, and
the navigator provides the navigation services during or after the communication session to assist the patient in participating in the clinical trial.
